(19) United States
(12) Patent Application Publication
(10) Pub. No.: US 2013/0281855 A1
(43) Pub. Date: Oct. 24, 2013

(54) ULTRASOUND DIAGNOSIS APPARATUS AND CONTROLLING METHOD THEREFOR

(71) Applicants: TOSHIBA MEDICAL SYSTEMS CORPORATION, Otawara-Shi (JP); KABUSHIKI KAISHA TOSHIBA, Tokyo (JP)

(72) Inventors: Tatsuro BABA, OTAWARA-SHI (JP); Shinichi HASHIMOTO, OTAWARA-SHI (JP)

(21) Appl. No.: 13/918,315

(22) Filed: Jun. 14, 2013

Related U.S. Application Data
(63) Continuation of application No. PCT/JP2011/079245, filed on Dec. 16, 2011.

(30) Foreign Application Priority Data
Dec. 16, 2010 (JP) 2010-280797

(51) Int. Cl. A61B 8/08; A61B 8/14; A61B 8/00

(52) U.S. Cl. CPC A61B 8/5246; A61B 8/463; A61B 8/14; A61B 8/488; A61B 8/54
USPC 600/441

(57) ABSTRACT

In an ultrasound diagnosis apparatus according to an embodiment, a scan switching unit switches between scanning methods so as to perform a first scanning method by which an ultrasound wave is transmitted and received to and from each of a plurality of observation sites alternately once each if the depth on the scanning line in at least one of the observation sites is smaller than a threshold value and so as to perform a second scanning method by which an ultrasound wave is transmitted and received to and from each of the plurality of observation sites alternately in such a manner that an ultrasound wave is transmitted and received to and from at least one of the plurality of observation sites multiple times if the depth on the scanning line in the at least one of the observation sites is equal to or larger than the threshold value.

### CROSS-REFERENCE TO RELATED APPLICATIONS

This application is a continuation of PCT international application Ser. No. PCT/JP2011/079245 filed on Dec. 16, 2011 which designates the United States, and which claims the benefit of priority from Japanese Patent Application No. 2010-280797, filed on Dec. 16, 2010; the entire contents of which are incorporated herein by reference.

### FIELD

Embodiments described herein relate generally to an ultrasound diagnosis apparatus and a controlling method therefor.

### BACKGROUND

An ultrasound diagnosis apparatus conventionally known is configured to set a range gate in a blood vessel image (e.g., a B-mode image) as a bloodstream information observation site and to display a Doppler spectrum image indicating chronological changes in the blood flow rate at the range gate. Also known is a dual Doppler technique by which such an ultrasound diagnosis apparatus displays Doppler spectrum images that respectively correspond to range gates set in a plurality of locations.

In this situation, examples of scanning methods used by the dual Doppler technique include an interleaved scanning method and a segment scanning method. The interleaved scanning method is a method by which bloodstream information at the range gates is obtained by transmitting and receiving an ultrasound wave to and from each of the range gates set in a plurality of locations, alternately once each. In contrast, the segment scanning method is a method by which bloodstream information at the range gates is obtained by transmitting and receiving an ultrasound wave to and from each of the range gates set in a plurality of locations, alternately multiple times each.

According to the conventional technique, however, there are some situations where it is not possible to obtain a satisfactory Doppler spectrum image due to a sound velocity limit of the ultrasound waves. For example, when the interleaved scanning method is used, because the velocity range is limited to a low level, there is a higher possibility that an aliasing phenomenon may occur in a Doppler spectrum image related to a fast bloodstream flowing in the depth of an examined subject (hereinafter, “patient”). In contrast, when the segment scanning method is used, while an ultrasound wave is being transmitted and received to and from one range gate successively, no ultrasound wave is transmitted and received to and from the other range gates. For this reason, periodical data absences occur in the Doppler spectrum images at the range gates, and the data absences can cause degradation in the images.

### BRIEF DESCRIPTION OF THE DRAWINGS

FIG. 1 is a block diagram of an ultrasound diagnosis apparatus according to an embodiment.

FIG. 2 is a block diagram of a functional configuration of a controlling unit according to the embodiment.

FIGS. 3A and 3B are drawings for explaining a single Doppler mode used in the ultrasound diagnosis apparatus according to the embodiment.

FIGS. 4A and 4B are drawings for explaining the single Doppler mode used in the ultrasound diagnosis apparatus according to the embodiment.

FIGS. 5A and 5B are drawings for explaining a dual Doppler mode used in the ultrasound diagnosis apparatus according to the embodiment.

FIGS. 6A and 6B are drawings for explaining the dual Doppler mode used in the ultrasound diagnosis apparatus according to the embodiment.

FIGS. 7A and 7B are drawings for explaining the dual Doppler mode used in the ultrasound diagnosis apparatus according to the embodiment.

FIG. 8 is a drawing for explaining a distance judging process performed by a distance judging unit according to the embodiment.

FIG. 9 is a chart of a sequence in an interleaved scanning method according to the embodiment.

FIG. 10 is a chart of a processing flow in the interleaved scanning method according to the embodiment.

FIG. 11 is a chart of a sequence in an interleaved scanning method performed when a left ventricular inflow early diastolic filling velocity and a mitral annular motion velocity are selected.

FIG. 12 is a chart of a processing flow in the interleaved scanning method performed when the left ventricular inflow early diastolic filling velocity and the mitral annular motion velocity are selected.

FIG. 13 is a chart of a sequence in a segment scanning method according to the embodiment.

FIG. 14 is a chart of a processing flow in a segment scanning method according to the embodiment.

FIG. 15 is a drawing for explaining a sound velocity limit of ultrasound waves.

FIG. 16 is a drawing of an exemplary display of measured values realized by a measured value display unit according to the embodiment.

FIG. 17 is a drawing of an example of a measured value calculation performed by a measured value calculating unit according to the embodiment.

FIG. 18 is a drawing of an exemplary display of measured values realized by the measured value display unit according to the embodiment.

FIGS. 19A, 19B, and 19C are drawings of examples of measured value calculations performed by the measured value calculating unit according to the embodiment.

FIG. 20 is a flowchart of a processing procedure in a B/D simultaneous scan performed by the ultrasound diagnosis apparatus according to the embodiment.

FIG. 21 is a flowchart of a processing procedure in an automatic measuring process performed by the ultrasound diagnosis apparatus according to the embodiment.

### DETAILED DESCRIPTION

An ultrasound diagnosis apparatus according to an embodiment includes a setting unit, a distance judging unit, a scan switching unit, an image generating unit, and a display unit. The setting unit is configured to set a plurality of observation sites. The distance judging unit is configured to compare a depth on a scanning line in at least one of the plurality of observation sites with a predetermined threshold value. The scan switching unit is configured to switch between scanning methods so as to perform a first scanning method by which an ultrasound wave is transmitted and received to and from each of the plurality of observation sites alternately once each if the depth on the scanning line in said at least one of the observation sites is smaller than the threshold value and so as to perform a second scanning method by which an ultrasound wave is transmitted and received to and from each of the plurality of observation sites alternately in such a manner that an ultrasound wave is transmitted and received to and from at least one of the plurality of observation sites multiple times if the depth on the scanning line in said at least one of the observation sites is equal to or larger than the threshold value. The image generating unit is configured to generate a Doppler spectrum image indicating a chronological change in a moving velocity at each of the plurality of observation sites, based on reflected-wave data received as a result of the first scanning method or the second scanning method. The display unit configured to display the Doppler spectrum images.

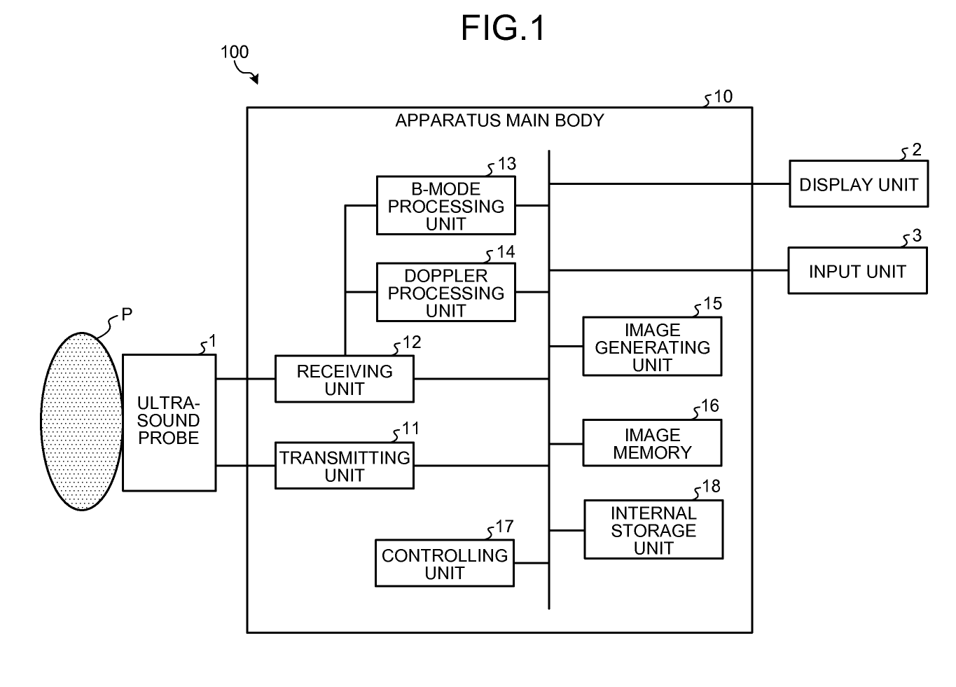

First, a configuration of an ultrasound diagnosis apparatus according to an embodiment will be explained. FIG. 1 is a block diagram of an ultrasound diagnosis apparatus 100 according to the present embodiment. As shown in FIG. 1, the ultrasound diagnosis apparatus 100 according to the present embodiment includes an ultrasound probe 1, a display unit 2, an input unit 3, and an apparatus main body 10.

The ultrasound probe 1 includes a plurality of piezoelectric transducers, which generate an ultrasound wave based on a drive signal supplied from a transmitting unit 11 included in the apparatus main body 10 (explained later). Further, the ultrasound probe 1 receives a reflected wave from a patient P and converts the received reflected wave into an electric signal. Further, the ultrasound probe 1 includes matching layers and acoustic lenses that are provided in the piezoelectric transducers, as well as a backing member that prevents ultrasound waves from propagating rearward from the piezoelectric transducers. The ultrasound probe 1 is detachably connected to the apparatus main body 10.

When an ultrasound wave is transmitted from the ultrasound probe 1 to the patient P, the transmitted ultrasound wave is repeatedly reflected on a surface of discontinuity of acoustic impedances at a tissue inside the body of the patient P and is received as a reflected-wave signal by the plurality of piezoelectric transducers included in the ultrasound probe 1. The amplitude of the received reflected-wave signal is dependent on the difference between the acoustic impedances on the surface of discontinuity on which the ultrasound wave is reflected. When the transmitted ultrasound pulse is reflected on the surface of a moving member such as a bloodstream or a cardiac wall, the reflected-wave signal is, due to the Doppler effect, subject to a frequency shift (a Doppler shift), depending on a velocity component of the moving members with respect to the ultrasound wave transmission direction.

The present embodiment is applicable to a situation where the patient P is scanned two-dimensionally by using the ultrasound probe 1 configured with a one-dimensional ultrasound probe in which the plurality of piezoelectric transducers are arranged in a row and is also applicable to a situation where the patient P is scanned three-dimensionally by using the ultrasound probe 1 configured so as to mechanically oscillate the plurality of piezoelectric transducers included in a one-dimensional ultrasound probe or by using the ultrasound probe 1 configured with a two-dimensional ultrasound probe in which the plurality of piezoelectric transducers are arranged two-dimensionally in a matrix formation.

The input unit 3 includes a mouse, a keyboard, a button, a panel switch, a touch command screen, a foot switch, a trackball, and the like. The input unit 3 receives various types of requests from an operator of the ultrasound diagnosis apparatus 100 and transfers the received various types of requests to the apparatus main body 10.

For example, by using the trackball included in the input unit 3, the operator sets one or more range gates each indicating a bloodstream information observation site, in a blood vessel image such as a B-mode image. Further, for example, by using the panel switch or the like included in the input unit 3, the operator makes a start request and an end request for a B/D simultaneous scanning by which a B-mode image and a Doppler spectrum image are displayed.

The display unit 2 displays a Graphical User Interface (GUI) used by the operator of the ultrasound diagnosis apparatus 100 to input the various types of requests through the input unit 3 and displays an ultrasound image generated by the apparatus main body 10.

The apparatus main body 10 generates the ultrasound image based on the reflected wave received by the ultrasound probe 1. More specifically, the apparatus main body 10 includes the transmitting unit 11, a receiving unit 12, a B-mode processing unit 13, a Doppler processing unit 14, an image generating unit 15, an image memory 16, a controlling unit 17, and an internal storage unit 18.

The transmitting unit 11 includes a trigger generating circuit, a transmission delaying circuit, and a pulser circuit and supplies the drive signal to the ultrasound probe 1. The pulser circuit repeatedly generates a rate pulse for forming a transmission ultrasound wave at a predetermined Pulse Repetition Frequency (PRF). The PRF may be called a rate frequency. The transmission delaying circuit applies a transmission delay period that is required to converge the ultrasound wave generated by the ultrasound probe 1 into the form of a beam and to determine transmission directionality and that corresponds to each of the piezoelectric transducers, to each of the rate pulses generated by the pulse circuit. Further, the trigger generating circuit applies a drive signal (a drive pulse) to the ultrasound probe 1 with timing based on the rate pulses. In other words, the transmission delaying circuit arbitrarily adjusts the directions of the transmissions from the piezoelectric transducer surfaces, by varying the transmission delay periods applied to the rate pulses.

The transmitting unit 11 has a function to be able to instantly change the transmission frequency, the transmission drive voltage, and the like, for the purpose of executing a predetermined scanning sequence based on an instruction from the controlling unit 17 (explained later). In particular, the configuration to change the transmission drive voltage is realized by using a linear-amplifier-type transmitting circuit of which the value can be instantly switched or by using a mechanism configured to electrically switch between a plurality of power source units.

In this situation, each of the transmission delay periods is determined depending on the position (the depth) of a transmission focus of the ultrasound beam with respect to the acoustic lens. Further, by using the transmission delay periods, the transmitting unit 11 controls the transmission directionalities in the transmissions of the ultrasound waves.

The receiving unit 12 includes an amplifying circuit, an Analog/Digital (A/D) converter, a reception delaying circuit, an adder, and the like and generates reflected-wave data by performing various types of processes on the reflected-wave signal received by the ultrasound probe 1. The amplifying circuit amplifies the reflected-wave signal for each of channels and performs a gain correcting process thereon. The A/D converter applies an A/D conversion to the gain-corrected reflected-wave signal. The reception delaying circuit applies a reception delay period required to determine reception directionality, to digital data. The adder generates the reflected-wave data by performing an adding process on reflected-wave signals to which the reception delay periods have been applied by the reception delaying circuit. As a result of the adding process performed by the adder, reflected components from the direction corresponding to the reception directionality of the reflected-wave signal are emphasized.

In this situation, each of the reception delay periods is determined depending on the position (the depth) of a reception focus of the ultrasound beam with respect to the acoustic lens. Further, by using the reception delay periods, the receiving unit 12 controls the reception directionalities in the receptions of the ultrasound waves.

Further, the ultrasound probe 1 according to the present embodiment is able to change which of the piezoelectric transducers are used for the transmissions and the receptions (a transmission diameter and a reception diameter), according to the positions of the transmission focus and the reception focus. For example, to receive reflected-wave signals from a nearby position, the quantity of transducers used for the reception is arranged to be small so that a strong reception focus is applied. A small reception diameter is thus determined as a reception condition so that only the reflected-wave signals received by the piezoelectric transducers positioned in a central part are used for the generation of an ultrasound image. In contrast, to receive reflected-wave signals from a distant position, a reception condition is determined by arranging the reception diameter to be larger according to the distance, because the larger the diameter formed by the piezoelectric transducers is, the stronger is the reception focus.

The B-mode processing unit 13 generates data (B-mode data) in which the strength of each signal is expressed by a degree of brightness, by performing a logarithmic amplification, an envelop detection process, and the like on the reflected-wave data generated by the receiving unit 12.

The Doppler processing unit 14 extracts a Doppler shift by performing a frequency analysis so as to obtain velocity information from the reflected-wave data generated by the receiving unit 12. By utilizing the Doppler shift, the Doppler processing unit 14 extracts bloodstreams, tissues, and contrast echo components under the influence of the Doppler effect and generates data (Doppler data) obtained by extracting moving member information such as an average velocity, the dispersion, the power, and the like for a plurality of points.

The B-mode processing unit 13 and the Doppler processing unit 14 according to the present embodiment are capable of processing both two-dimensional reflected-wave data and three-dimensional reflected-wave data.

The image generating unit 15 generates an ultrasound image from the data generated by the B-mode processing unit 13 and the Doppler processing unit 14. More specifically, from the B-mode data generated by the B-mode processing unit 13, the image generating unit 15 generates the B-mode image in which the strength of the reflected wave is expressed by a degree of brightness. Alternatively, from the B-mode data that was generated by the B-mode processing unit 13 and that corresponds to a predetermined scanning line, the image generating unit 15 generates an M-mode image in which changes in the strength of the reflected wave in a time series for the predetermined scanning line is expressed by a degree of brightness.

Further, from the Doppler data generated by the Doppler processing unit 14, the image generating unit 15 generates an average velocity image, a dispersion image, and a power image, expressing moving member information (bloodstream information and/or tissue movement information), or a color Doppler image, which is a image combining these images. Further, from the Doppler data generated by the Doppler processing unit 14, the image generating unit 15 generates a Doppler spectrum image in which the velocity information of the moving members (velocity information of the bloodstream and/or velocity information of the tissue) is plotted along the time series.

The image memory 16 is a memory storing therein the ultrasound image generated by the image generating unit 15. Further, the image memory 16 is also able to store therein the data generated by the B-mode processing unit 13 and the Doppler processing unit 14.

The internal storage unit 18 stores therein various types of data such as a control computer program to realize ultrasound transmissions and receptions, image processing, and display processing, as well as diagnosis information (e.g., patients' IDs, medical doctors' observations), diagnosis protocols, and various types of body marks. Further, the internal storage unit 18 may be used, as necessary, for storing therein any of the images stored in the image memory 16. Furthermore, the data stored in the internal storage unit 18 may be transferred to any external peripheral device via an interface unit (not shown).

The controlling unit 17 controls the entire processes performed by the ultrasound diagnosis apparatus 100. More specifically, based on the various types of requests input by the operator via the input unit 3 and various types of control computer programs and various types of data read from the internal storage unit 18, the controlling unit 17 controls processes performed by the transmitting unit 11, the receiving unit 12, the B-mode processing unit 13, the Doppler processing unit 14, and the image generating unit 15. The controlling unit 17 also exercises control so that the ultrasound images stored in the image memory 16 and a GUI used for specifying various types of processes performed by the image generating unit 15 are displayed on the display unit 2.

A configuration of the ultrasound diagnosis apparatus 100 according to the present embodiment has thus been explained. In the ultrasound diagnosis apparatus 100 according to the present embodiment configured as described above, with respect to at least two range gates set as bloodstream information observation sites, the controlling unit 17 judges whether a total length of the distance from a first range gate to the ultrasound probe and the distance from a second range gate to the ultrasound probe is smaller than a threshold value. Further, the controlling unit 17 switches between the scanning methods so as to perform an interleaved scanning method if the total length of the distances is determined to be smaller than the threshold value and so as to perform a segment scanning method if the total length of the distances is determined to be equal to or larger than the threshold value. Further, based on reflected-wave data received as a result of the segment scanning method or the interleaved scanning method, the image generating unit 15 generates a first Doppler spectrum image indicating chronological changes in the blood flow rate at the first range gate and a second Doppler spectrum image indicating chronological changes in the blood flow rate at the second range gate. Further, the display unit 2 displays the first Doppler spectrum image and the second Doppler spectrum image generated by the image generating unit 15.

In other words, to display the Doppler spectrum images that respectively correspond to the range gates set in the plurality of locations, the ultrasound diagnosis apparatus 100 according to the present embodiment automatically switches between the interleaved scanning method and the segment scanning method according to the total of the depths of the range gates. In this situation, the interleaved scanning method is a method by which an ultrasound wave is transmitted and received to and from each of the first and the second range gates, alternately once each. In contrast, the segment scanning method is a method by which an ultrasound wave is transmitted and received to and from each of the first and the second range gates, alternately multiple times each.

In the following sections, the ultrasound diagnosis apparatus 100 configured in this manner will be explained in detail. In the present embodiment, the ultrasound diagnosis apparatus 100 is configured to set two range gates as the bloodstream information observation sites in a blood vessel image within the B-mode image and to display Doppler spectrum images respectively corresponding to the range gates. A display mode in which the Doppler spectrum images respectively corresponding to the two range gates are displayed in this manner will be hereinafter referred to as a “dual Doppler mode”. The ultrasound diagnosis apparatus 100 is also able to display each of the Doppler spectrum images respectively corresponding to the two range gates, one at a time. A display mode in which each of the Doppler spectrum images respectively corresponding to the two range gates is displayed one at a time in this manner will be hereinafter referred to as a “single Doppler mode”.

Further, the ultrasound diagnosis apparatus 100 is able to execute various types of applications according to the organ selected as a diagnosis target and the type of diagnosis to be made. In the present embodiment, examples will be explained in which the ultrasound diagnosis apparatus 100 executes an application for making a diagnosis of the heart and an application for making a diagnosis of the carotid arteries. Further, the ultrasound diagnosis apparatus 100 is able to switch between display modes of Doppler spectrum images according to the sites on which a diagnosis is to be made (hereinafter, “diagnosed sites”). In the present embodiment, the following examples will be explained: an example in which a Left Ventricular Inflow (LVI) and a Left Ventricular Outflow (LVO) of the heart are the diagnosed sites; an example in which a Left Ventricular Inflow early diastolic filling velocity (E) and a mitral annular motion velocity (e′) of the heart are the diagnosed sites; and an example in which the Common Carotid Artery (CCA) and the Internal Carotid Artery (ICA) among the carotid arteries are the diagnosed sites.

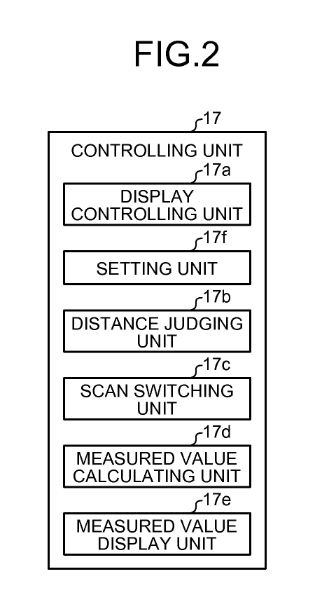

Next, the controlling unit 17 according to the present embodiment will be explained in detail. FIG. 2 is a block diagram of a functional configuration of the controlling unit 17 according to the present embodiment. As shown in FIG. 2, the controlling unit 17 includes a display controlling unit 17a, a setting unit 17f, a distance judging unit 17b, a scan switching unit 17c, a measured value calculating unit 17d, and a measured value display unit 17e.

The display controlling unit 17a receives various types of requests from the operator via the input unit 3 and causes the display unit 2 to display, according to the received various types of requests, any of the ultrasound images stored in the image memory 16 and the GUI used for specifying various types of processes performed by the image generating unit 15. Further, via the touch command screen included in the input unit 3, the ultrasound diagnosis apparatus 100 receives operations to select a display mode, an application, and a diagnosed site as described above, from the operator.

For example, the display controlling unit 17a causes a “Dual Doppler” button, a “PWD1” button, and a “PWD2” button to be displayed on the touch screen. The “Dual Doppler” button is a button used for receiving a selection out of the single mode and the dual mode and a selection of a diagnosed site, from the operator. Every time the operator presses the “Dual Doppler”, the display thereof sequentially changes as follows: “Dual Doppler (off)”, “Dual Doppler (LVI/LVO)”, and “Dual Doppler (E/e′)”.

The “PWD1” button and the “PWD2” button are buttons used for receiving an operation to select one of the two range gates, from the operator. The “PWD1” button and the “PWD2” button are displayed as “PWD1” and “PWD2” while the “Dual Doppler” button is displayed as “Dual Doppler (off)”, are displayed as “PWD1 (LVI)” and “PWD2 (LVO)” while the “Dual Doppler” button is displayed as “Dual Doppler (LVI/LVO)”, and are displayed as “PWD1 (E)” and “PWD2 (e′)” while the “Dual Doppler” button is displayed as “Dual Doppler (E/e′)”.

Further, for example, when having received a B/D simultaneous scan start request from the operator, the display controlling unit 17a causes the display unit 2 to display the B-mode image and the Doppler spectrum image generated by the image generating unit 15. Further, in the B-mode image displayed on the display unit 2, the display controlling unit 17a displays two scanning lines indicating the transmission/reception directions of the ultrasound waves. In addition, the display controlling unit 17a displays a range gate on each of the scanning lines. Further, according to an operation received from the operator via the trackball included in the input unit 3, the display controlling unit 17a moves each of the scanning lines in a scanning direction and moves the position of each of the range gates along the scanning line.

In this situation, according to the display mode, the application, and the diagnosed site selected by the operator, the display controlling unit 17a changes the positions of the scanning lines and the range gates displayed in the B-mode image and the type of the Doppler spectrum image. For example, the positions of the scanning lines and the range gates are determined based on preset information that is defined in advance for each of the applications and the diagnosed sites.

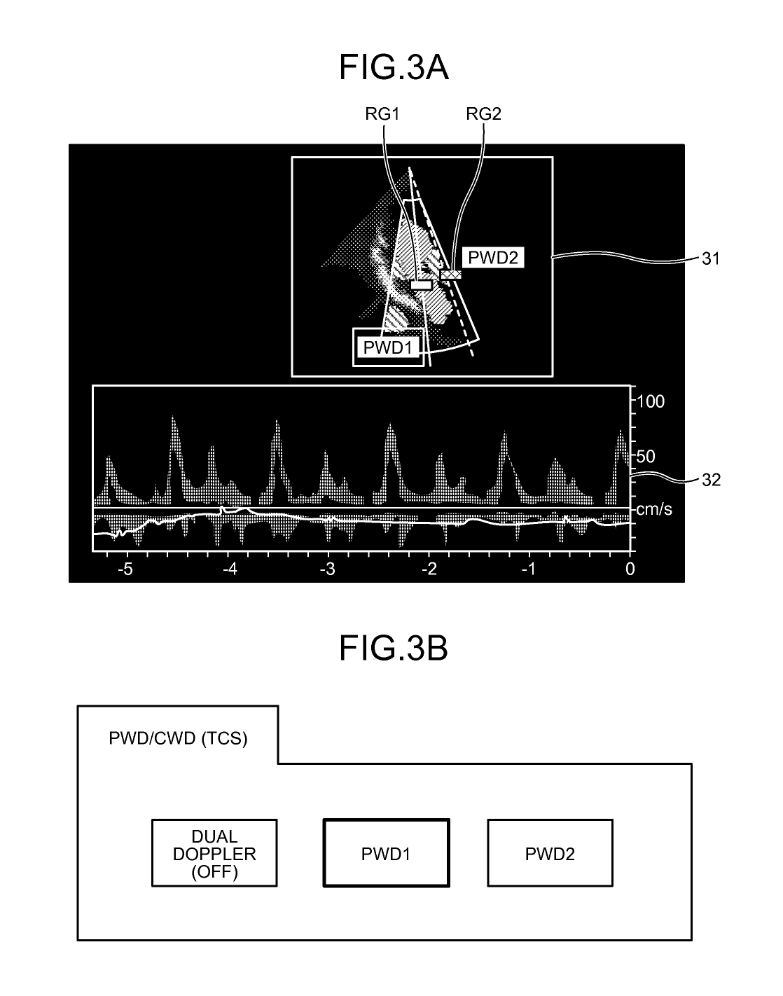

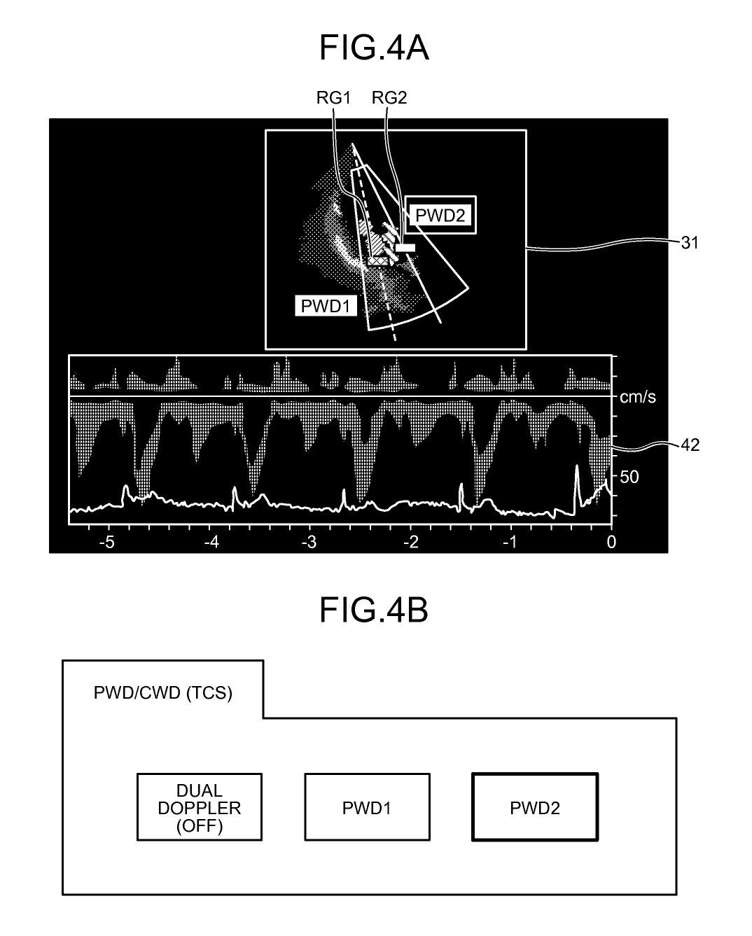

FIGS. 3A, 3B, 4A, and 4B are drawings for explaining the single Doppler mode used in the ultrasound diagnosis apparatus 100 according to the present embodiment. Shown in FIGS. 3A, 3B, 4A, and 4B are examples in which an application for making a diagnosis of the heart is selected, and the left ventricular inflow and the left ventricular outflow are selected as the diagnosed sites. A display area of the display unit 2 is shown in FIGS. 3A and 4A, whereas the touch command screen is shown in FIGS. 3B and 4B.

As shown in FIGS. 3A, 3B, 4A, and 4B, when the application for making a diagnosis of the heart is selected, and the left ventricular inflow and the left ventricular outflow are selected as the diagnosed sites, the display controlling unit 17a displays a B-mode image 31 on the display unit 2 and displays two scanning lines PWD1 and PWD2 in the B-mode image 31. Further, the display controlling unit 17a displays a range gate RG1 on the scanning line PWD1 and displays a range gate RG2 on the scanning line PWD2. In this situation, the display controlling unit 17a displays the scanning lines PWD1 and PWD2 and the range gates RG1 and RG2 in such a manner that the range gate RG1 is arranged in the position of the left ventricular inflow, whereas the range gate RG2 is arranged in the position of the left ventricular outflow.

Further, for example, as shown in FIGS. 3A and 3B, when the “PWD1” button is pressed while the “Dual Doppler” button is displayed as “Dual Doppler (off)” on the touch command screen, the display controlling unit 17a causes a Doppler spectrum image 32 at the range gate RG1 set on the scanning line PWD1 to be displayed in the display area of the display unit 2. In this situation, the display controlling unit 17a arranges the display in such a manner that it is possible to receive operations performed on the scanning line PWD1 and the range gate RG1.

In another example, as shown in FIGS. 4A and 4B, when the “PWD2” button is selected while the “Dual Doppler” button is displayed as “Dual Doppler (off)” on the touch command screen, the display controlling unit 17a causes a Doppler spectrum image 42 at the range gate RG2 set on the scanning line PWD2 to be displayed in the display area of the display unit 2. In this situation, the display controlling unit 17a arranges the display in such a manner that it is possible to receive operations performed on the scanning line PWD2 and the range gate RG2.

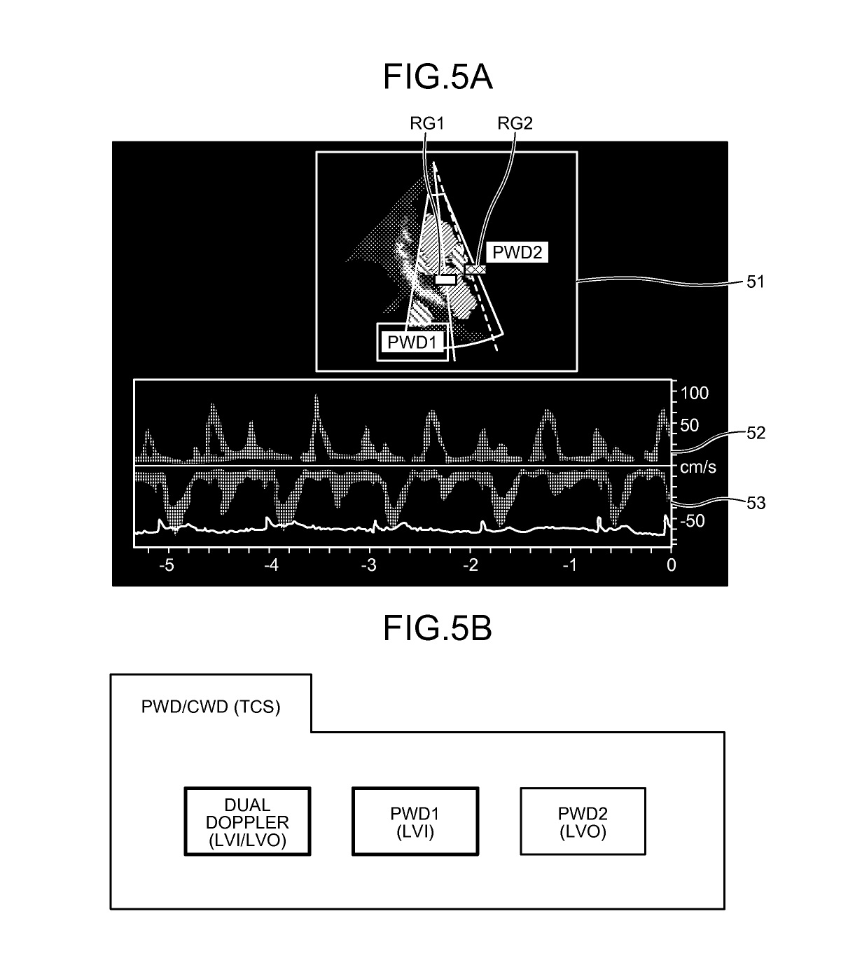

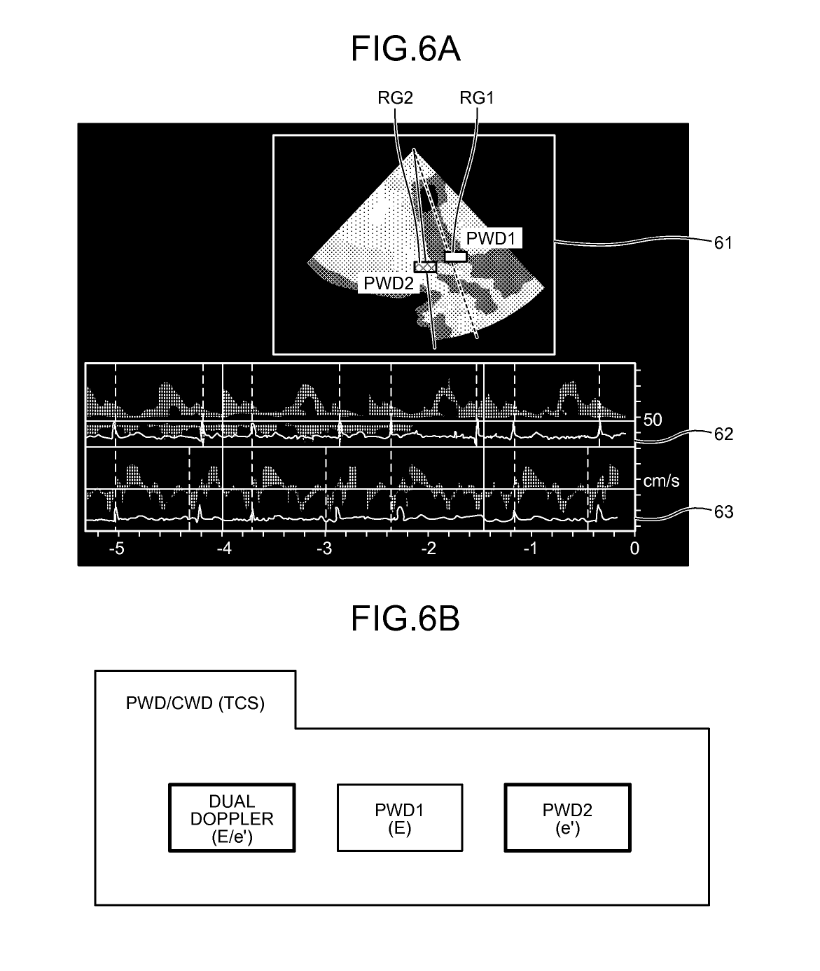

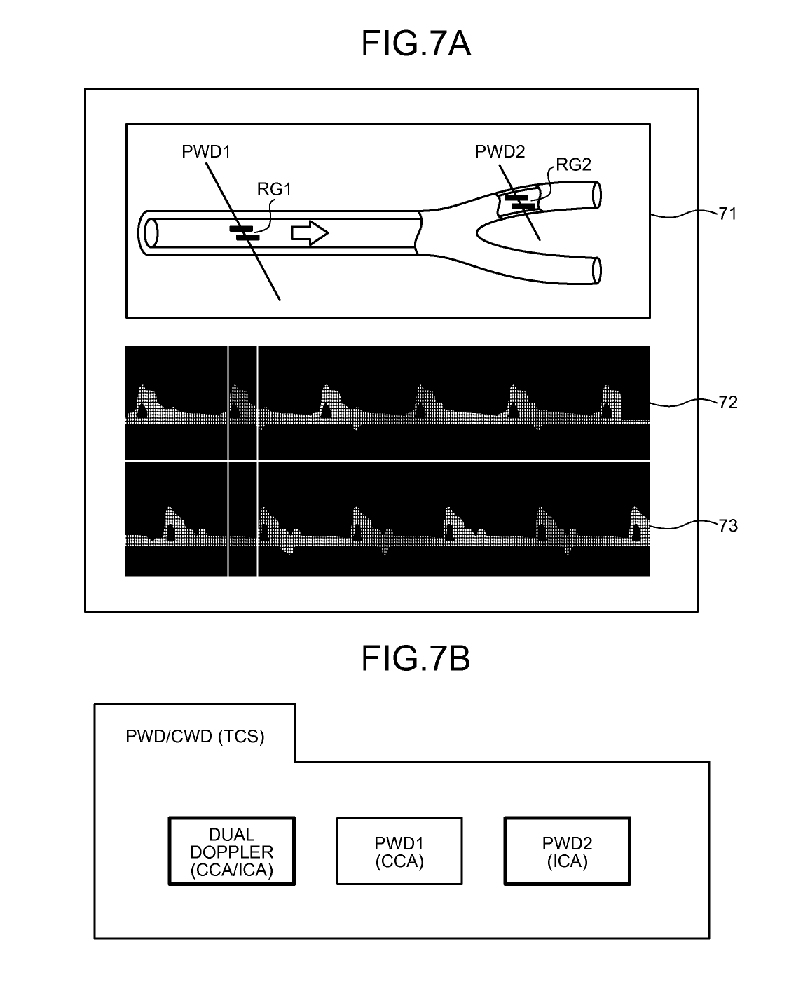

FIGS. 5A, 5B, 6A, 6B, 7A, and 7B are drawings for explaining the dual Doppler mode used in the ultrasound diagnosis apparatus 100 according to the present embodiment. Shown in FIGS. 5A and 5B is an example in which an application for making a diagnosis of the heart is selected, and the left ventricular inflow and the left ventricular outflow are selected as the diagnosed sites. Shown in FIGS. 6A and 6B is an example in which an application for making a diagnosis of the heart is selected, and a left ventricular inflow early diastolic filling velocity and a mitral annular motion velocity are selected as the diagnosed sites. Further, shown in FIGS. 7A, and 7B is an example in which an application for making a diagnosis of the carotid arteries is selected, and the common carotid artery and the internal carotid artery are selected as the diagnosed sites.

As shown in FIGS. 5A and 5B, when the application for making a diagnosis of the heart is selected, and the left ventricular inflow and the left ventricular outflow are selected as the diagnosed sites, the display controlling unit 17a displays a B-mode image 51 of the heart on the display unit 2 and displays two scanning lines PWD1 and PWD2 in the B-mode image 51. Further, the display controlling unit 17a displays a range gate RG1 on the scanning line PWD1 and displays a range gate RG2 on the scanning line PWD2. In this situation, the display controlling unit 17a displays the scanning lines PWD1 and PWD2 and the range gates RG1 and RG2 in such a manner that the range gate RG1 is arranged in the position of the left ventricular inflow, whereas the range gate RG2 is arranged in the position of the left ventricular outflow.

Further, for example, as shown in FIGS. 5A and 5B, when the “Dual Doppler” button is displayed as “Dual Doppler (LVI/LVO)” on the touch command screen, the display controlling unit 17a causes a Doppler spectrum image 52 indicating a velocity component on the positive side at the range gate RG1 and a Doppler spectrum image 53 indicating a velocity component on the negative side at the range gate RG2 to be displayed one above the other, in the display area of the display unit 2. If the “PWD1” button is pressed in this situation, the display controlling unit 17a arranges the display in such a manner that it is possible to receive operations performed on the scanning line PWD1 and the range gate RG1. If the “PWD2” button is pressed, the display controlling unit 17a arranges the display in such a manner that it is possible to receive operations performed on the scanning line PWD2 and the range gate RG2.

In another example, as shown in FIGS. 6A and 6B, when the application for making a diagnosis of the heart is selected, and the left ventricular inflow early diastolic filling velocity and the mitral annular motion velocity are selected as the diagnosed sites, the display controlling unit 17a displays a B-mode image 61 of the heart on the display unit 2 and displays two scanning lines PWD1 and PWD2 in the B-mode image 61. Further, the display controlling unit 17a displays a range gate RG1 on the scanning line PWD1 and displays a range gate RG2 on the scanning line PWD2. In this situation, the display controlling unit 17a displays the scanning lines PWD1 and PWD2 and the range gates RG1 and RG2 in such a manner that the range gate RG1 is arranged in the position of the left ventricular inflow, whereas the range gate RG2 is arranged in the position of the mitral valve ring.

Further, for example, as shown in FIGS. 6A and 6B, when the “Dual Doppler” button is displayed as “Dual Doppler (E/e′)” on the touch command screen, the display controlling unit 17a causes a Doppler spectrum image 62 indicating the left ventricular inflow early diastolic filling velocity at the range gate RG1 and a Doppler spectrum image 63 indicating the mitral annular motion velocity to be displayed one above the other, in the display area of the display unit 2. If the “PWD1” button is pressed in this situation, the display controlling unit 17a arranges the display in such a manner that it is possible to receive operations performed on the scanning line PWD1 and the range gate RG1. If the “PWD2” button is pressed, the display controlling unit 17a arranges the display in such a manner that it is possible to receive operations performed on the scanning line PWD2 and the range gate RG2.

In another example, as shown in FIGS. 7A and 7B, when the application for making a diagnosis of the carotid arteries is selected, and the common carotid artery and the internal carotid artery are selected as the diagnosed sites, the display controlling unit 17a displays a B-mode image 71 of the carotid arteries on the display unit 2 and displays two scanning lines PWD1 and PWD2 in the B-mode image 71. Further, the display controlling unit 17a displays a range gate RG1 on the scanning line PWD1 and displays a range gate RG2 on the scanning line PWD2. In this situation, the display controlling unit 17a displays the scanning lines PWD1 and PWD2 and the range gates RG1 and RG2 in such a manner that the range gate RG1 is arranged in the position of the common carotid artery, whereas the range gate RG2 is arranged in the position of the internal carotid artery.

Further, for example, as shown in FIGS. 7A and 7B, when the “Dual Doppler” button is displayed as “Dual Doppler (CCA/ICA)” on the touch command screen, the display controlling unit 17a causes a Doppler spectrum image 72 of the common carotid artery at the range gate RG1 and a Doppler spectrum image 73 of the internal carotid artery to be displayed one above the other, in the display area of the display unit 2. If the “PWD1” button is pressed in this situation, the display controlling unit 17a arranges the display in such a manner that it is possible to receive operations performed on the scanning line PWD1 and the range gate RG1. If the “PWD2” button is pressed, the display controlling unit 17a arranges the display in such a manner that it is possible to receive operations performed on the scanning line PWD2 and the range gate RG2.

Returning to the description of FIG. 2, the setting unit 17f sets a plurality of observation sites. In the present embodiment, the setting unit 17f sets the observation sites, based on the positions of the range gates displayed on the display unit 2 by the display controlling unit 17a. More specifically, the setting unit 17f sets the locations in which the range gates are positioned within the B-mode image displayed on the display unit 2, as the observation sites.

The distance judging unit 17b compares the depth on the scanning line in at least one of the plurality of observation sites with a predetermined threshold value. In the present embodiment, the distance judging unit 17b compares a total of the depths on the scanning lines in at least two of the plurality of observation sites with a predetermined threshold value.

More specifically, with respect to at least two range gates that are set as bloodstream information observation sites, the distance judging unit 17b judges whether the total length of the distance from a first range gate to the ultrasound probe and the distance from a second range gate to the ultrasound probe and is smaller than the threshold value.

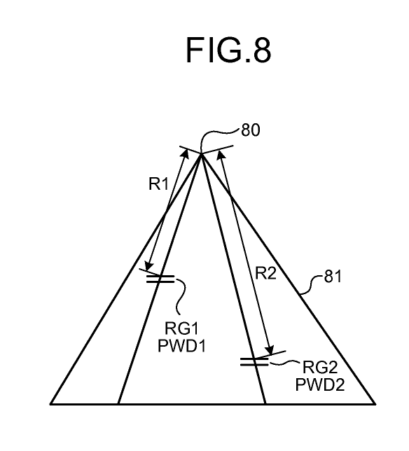

FIG. 8 is a drawing for explaining the distance judging process performed by the distance judging unit 17b according to the present embodiment. As shown in FIG. 8, let us assume that, for example, two scanning lines PWD1 and PWD2 are set in a B-mode image 81, while a range gate RG1 is set on the scanning line PWD1, whereas a range gate RG2 is set on the scanning line PWD2. In this situation, the distance judging unit 17b calculates a distance R1 from a probe origin 80 of the ultrasound probe 1 to the range gate RG1 and a distance R2 from the probe origin 80 of the ultrasound probe 1 to the range gate RG2. Further, the distance judging unit 17b calculates the total length of the calculated distances R1 and R2 and judges whether the total length is smaller than the predetermined threshold value.

In this situation, according to the present embodiment, the distance judging unit 17b makes the judgment on the total length of the distances by setting the threshold value based on the diagnosed sites. For example, when the left ventricular inflow and the left ventricular outflow of the heart are the diagnosed sites, the distance judging unit 17b sets a value twice as large as the depth of the range gate that causes no aliasing even if an interleaved scanning method is performed, as the threshold value. In this situation, to learn the depth of the range gate that causes no aliasing, for example, an interleaved scanning method may be experimentally performed in advance while gradually increasing the depth of the range gate, so as to set the threshold value to be smaller than the depth of the range gate at the point in time when aliasing occurs in the Doppler spectrum image. The threshold value learned from the depth is, for example, stored into a predetermined storage unit by the operator in advance. Further, the distance judging unit 17b obtains the threshold value stored in the storage unit and makes the judgment on the total length of the distances. When the left ventricular inflow early diastolic filling velocity and the mitral annular motion velocity of the heart are the diagnosed sites, a threshold value can be set in the same manner.

As another example, when the common carotid artery and the internal carotid artery among the carotid arteries are the diagnosed sites, the distance judging unit 17b sets a value larger than twice the possible maximum value of the depth of the range gate, as the threshold value. With this arrangement, when a diagnosis is to be made on the common carotid artery and the internal carotid artery among the carotid arteries, the data will be always acquired by performing an interleaved scanning method because there is no possibility that the total length of the distances from each of the range gates to the ultrasound probe becomes equal to or larger than the threshold value. Generally speaking, because the carotid arteries are positioned in shallow positions with respect to the surface of the body, the possibility of occurrence of an aliasing phenomenon is low even if the data is acquired by performing an interleaved scanning method. Consequently, it is possible to obtain a Doppler image having a sufficient level of image quality.

The examples in which the threshold values are set based on the diagnosed sites are explained above; however, the distance judging unit 17b may set a threshold value based on, for instance, patient information. For example, the distance judging unit 17b may set a threshold value based on the gender and the age of the patient that are input to the ultrasound diagnosis apparatus 100 by the operator, when a diagnosis is to be made. For example, it is known that the Doppler velocity range becomes lower, as the patient's age increases. For this reason, for example, the distance judging unit 17b sets threshold values in such a manner that the older the patient is, the smaller is the threshold value.

Returning to the description of FIG. 2, the scan switching unit 17c switches between the scanning methods so as to perform a first scanning method by which an ultrasound wave is transmitted and received to and from each of the plurality of observation sites alternately once each, if the depth on the scanning line in at least one of the observation sites is smaller than the threshold value and so as to perform a second scanning method by which an ultrasound wave is transmitted and received to and from each of the plurality of observation sites alternately in such a manner that an ultrasound wave is transmitted and received to and from at least one of the plurality of observation sites multiple times, if the depth on the scanning line in at least one of the observation sites is equal to or larger than the threshold value.

According to the present embodiment, the scan switching unit 17c switches between the scanning methods so as to perform the first scanning method if the total of the depths on the scanning lines in at least two observation sites is smaller than the threshold value and so as to perform the second scanning method if the total of the depths on the scanning lines in at least two observation sites is equal to or larger than the threshold value.

To perform the second scanning method, for example, an ultrasound wave may be transmitted and received to and from each of the plurality of observation sites an equal number of times or an ultrasound wave may be transmitted and received to and from each of the plurality of observation sites mutually-different numbers of times. For example, to perform the second scanning method, an arrangement is acceptable in which an ultrasound wave is transmitted to and from each of one or more of the plurality of observation sites once each, whereas an ultrasound wave is transmitted to and from each of the other observation sites multiple times.

In this situation, how many times an ultrasound wave is transmitted and received to and from each of the observation sites is determined according to, for example, the depth of the observation site. Generally speaking, the deeper one of the observation sites is positioned, the larger is the gap appearing in the Doppler waveform related to the other observation site, because it takes a longer period of time to transmit and receive an ultrasound wave to and from the one of the observation sites multiple times. To cope with this situation, the number of times an ultrasound wave is transmitted and received to and from an observation site in a deep position is arranged to be smaller than the number of times an ultrasound wave is transmitted and received to and from an observation site in a shallow position.

Further, how many times an ultrasound wave is transmitted and received may be determined according to, for example, a required precision level in the measuring process. For example, for an observation site that requires a higher precision level in the measuring process and for an observation site having a lower S/N ratio, an ultrasound wave may be transmitted and received a larger number of times. Further, how many times an ultrasound wave is transmitted and received may be determined, for example, according to a flow rate. For example, when the flow rate of an observation site is lower, an ultrasound wave is transmitted and received to and from the observation site once each, whereas an ultrasound wave is transmitted and received to and from each of the other observation sites multiple times. As a result, because the ultrasound wave is transmitted and received to and from the observation site having the lower flow rate at longer time intervals, it is possible to detect the lower flow rate.

More specifically, the scan switching unit 17c switches between the scanning methods so as to perform an interleaved scanning method if the distance judging unit 17b has determined that the total length of the distances is smaller than the threshold value and so as to perform a segment scanning method if the distance judging unit 17b has determined that the total length of the distances is equal to or larger than the threshold value. Next, the interleaved scanning method and the segment scanning method will be explained more specifically. In the following sections, an example will be explained in which data is acquired from the range gates RG1 and RG2 shown in FIG. 8.

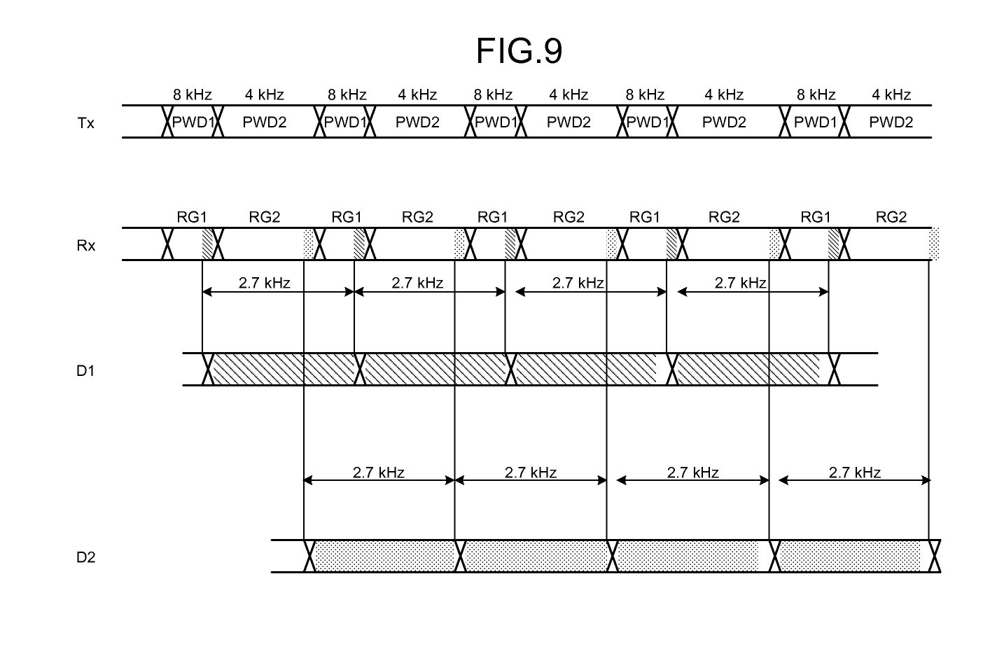

First, the interleaved scanning method will be explained. FIG. 9 is a chart of a sequence in an interleaved scanning method according to the present embodiment. In FIG. 9, the horizontal axis expresses time. Tx indicates the PRF and the transmission timing of the ultrasound wave transmitted from the ultrasound probe 1. Rx indicates the timing with which the reflected wave is received by the ultrasound probe 1. D1 indicates the timing with which the data for a Doppler spectrum image at the range gate RG1 is sampled. D2 indicates the timing with which the data for a Doppler spectrum image at the range gate RG2 is sampled.

According to the interleaved scanning method, an ultrasound wave is transmitted and received to and from each of the range gates RG1 and RG2, alternately once each. For example, as shown in FIG. 9, during the interleaved scanning method, an ultrasound wave having a PRF of 8 kilohertz is transmitted along the scanning line PWD1, whereas an ultrasound wave having a PRF of 4 kilohertz is transmitted along the scanning line PWD2. In this situation, the transmission to the scanning line PWD1 and the transmission to the scanning line PWD2 are performed alternately once each.

Further, during the interleaved scanning method, for example, the reflected wave from the range gate RG1 and the reflected wave from the range gate RG2 are received alternately. Further, for example, the data for a Doppler spectrum image at the range gate RG1 and the data for a Doppler spectrum image at the range gate RG2 are each sampled at a frequency of 2.7 kilohertz. To perform an interleaved scanning method, the PRFs are set to such values that make it possible to acquire the data from each of the range gates in the shortest period of time, according to the positions of the range gates RG1 and RG2.

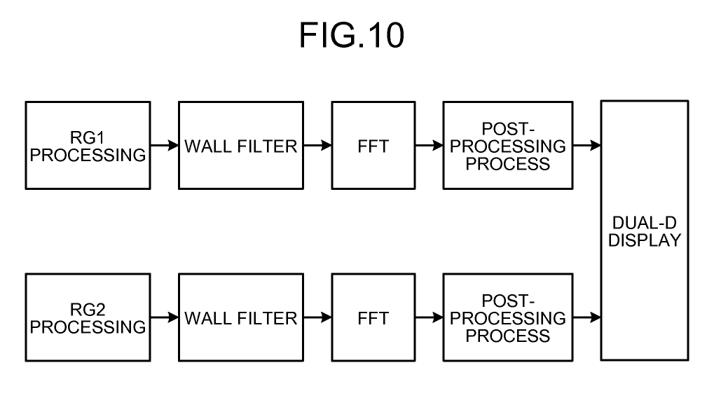

FIG. 10 is a chart of a processing flow in the interleaved scanning method according to the present embodiment. As shown in FIG. 10, during the interleaved scanning method, the Doppler processing unit 14 generates Doppler data indicating a blood flow rate at the range gate RG1 by sequentially applying a wall filter, a Fast Fourier transformation (FFT), and a post-processing process to the reflected-wave data from the range gate RG1.

Also, the Doppler processing unit 14 generates Doppler data indicating a blood flow rate at the range gate RG2 by sequentially applying a wall filter, a Fast Fourier transformation (FFT), and a post-processing process to the reflected-wave data from the range gate RG2. After that, from the pieces of Doppler data generated by the Doppler processing unit 14, the image generating unit 15 generates a Doppler spectrum image at the range gate RG1 and a Doppler spectrum image at the range gate RG2 and causes the display unit 2 to display the generated images (Dual-D display).

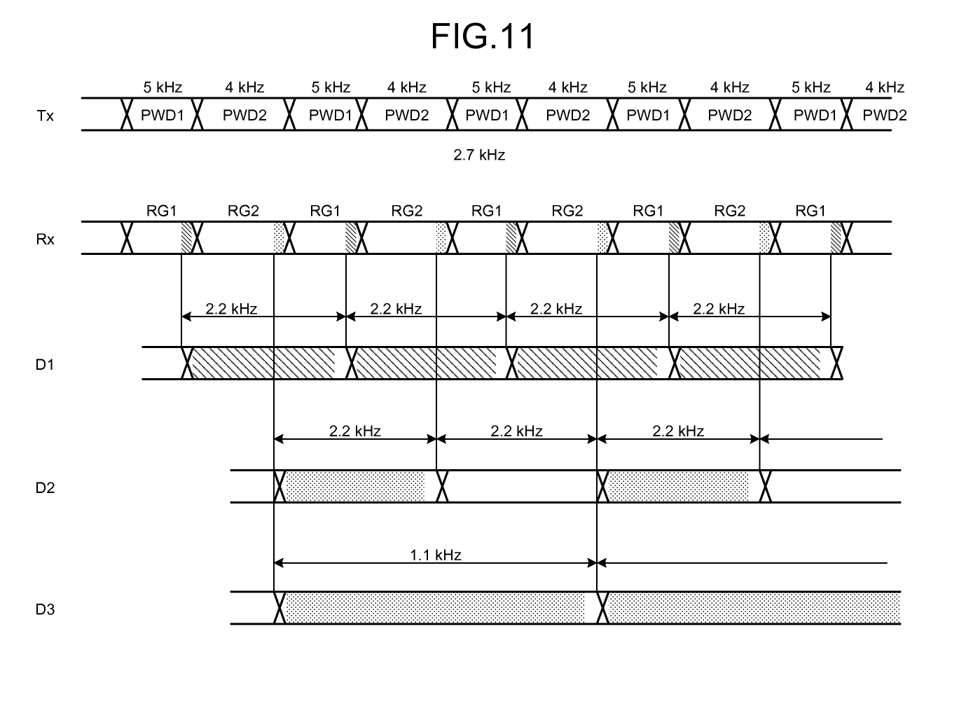

When an interleaved scanning method is performed after a left ventricular inflow early diastolic filling velocity and a mitral annular motion velocity of the heart are selected as the diagnosed sites, because the Doppler spectrum image representing the mitral annular motion velocity is generated according to a tissue Doppler method, the sequence is slightly different from the sequence shown in FIG. 9. FIG. 11 is a chart of a sequence in an interleaved scanning method performed when the left ventricular inflow early diastolic filling velocity and the mitral annular motion velocity are selected. In the present example, let us assume that a range gate RG1 is arranged in the position of the left ventricular inflow, whereas a range gate RG2 is arranged in the position of the mitral valve ring. In FIG. 11, the horizontal axis expresses time. Tx, Rx, D1, and D2 each indicate the same as in FIG. 9. D3 indicates the timing with which the data for the Doppler spectrum image representing the mitral annular motion velocity at the range gate RG2 is sampled.

During an interleaved scanning method performed when the left ventricular inflow early diastolic filling velocity and the mitral annular motion velocity are selected, an ultrasound wave is transmitted and received to and from each of the range gates RG1 and RG2 alternately multiple times each. For example, as shown in FIG. 11, during the interleaved scanning method, an ultrasound wave having a PRF of 5 kilohertz is transmitted along the scanning line PWD1, whereas an ultrasound wave having a PRF of 4 kilohertz is transmitted along the scanning line PWD2. In this situation, the transmission to the scanning line PWD1 and the transmission to the scanning line PWD2 are performed alternately once each.

Further, during the interleaved scanning method, for example, the reflected wave from the range gate RG1 and the reflected wave from the range gate RG2 are received alternately. Further, for example, the data for a Doppler spectrum image at the range gate RG1 and the data for a Doppler spectrum image at the range gate RG2 are each sampled at a frequency of 2.2 kilohertz. The data for a Doppler spectrum image representing the mitral annular motion velocity at the range gate RG2 is acquired while thinning the data, for example, at a frequency of 1.1 kilohertz. The reason is that the moving velocity of a tissue is lower than blood flow rates. To perform an interleaved scanning method, the PRFs are set to such values that make it possible to acquire the data from each of the range gates in the shortest period of time, according to the positions of the range gates RG1 and RG2.

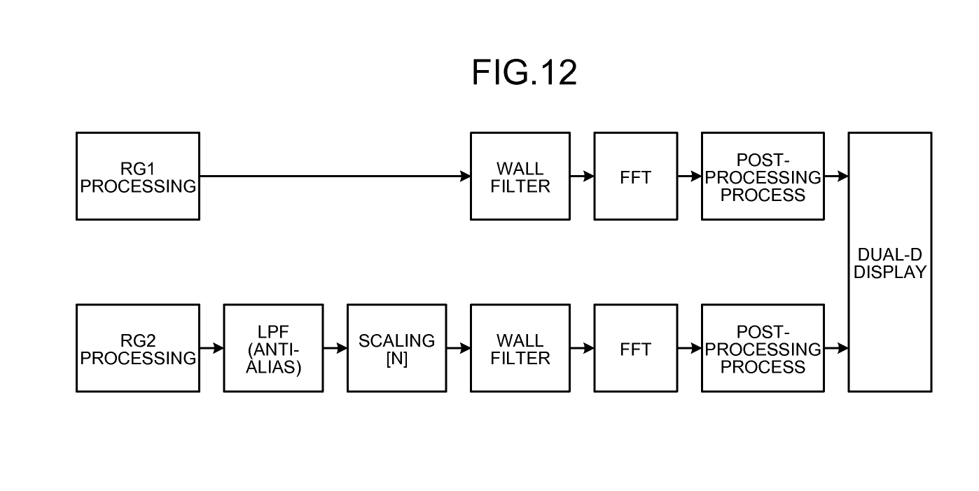

FIG. 12 is a chart of a processing flow in the interleaved scanning method performed when the left ventricular inflow early diastolic filling velocity and the mitral annular motion velocity are selected. As shown in FIG. 12, during the interleaved scanning method performed when the left ventricular inflow early diastolic filling velocity and the mitral annular motion velocity are selected, before applying a wall filter, the Doppler processing unit 14 applies a Low Pass Filter (LPF) and a scaling process to the reflected-wave data from the range gate RG2. As a result, the data for the Doppler spectrum image representing the mitral annular motion velocity is thinned.

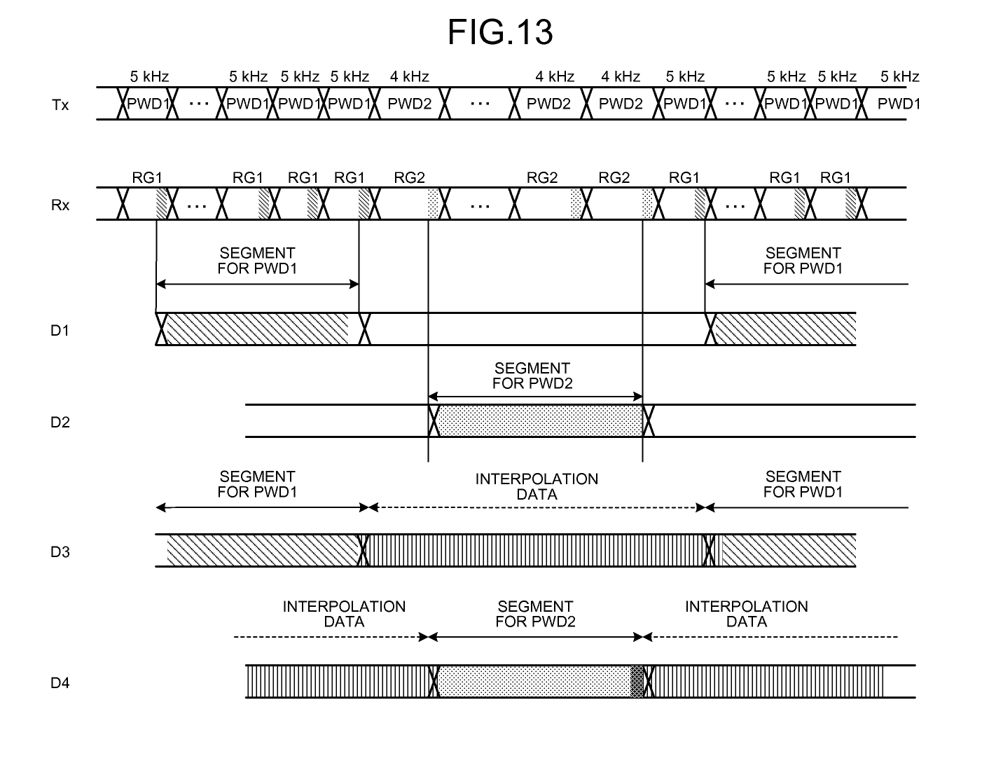

Next, the segment scanning method will be explained. FIG. 13 is a chart of a sequence in a segment scanning method according to the present embodiment. In FIG. 13, the horizontal axis expresses time. Further, Tx and Rx each indicate the same as in FIG. 9. D1 indicates the timing with which the data for a Doppler spectrum image at the range gate RG1 is sampled. D2 indicates the timing with which the data for a Doppler spectrum image at the range gate RG2 is sampled. D3 indicates signal processing related to the data for the Doppler spectrum image at the range gate RG1. D4 indicates signal processing related to the data for the Doppler spectrum image at the range gate RG2.

During a segment scanning method, an ultrasound wave is transmitted and received to and from each of the range gates RG1 and RG2 alternately multiple times each. For example, as shown in FIG. 13, during the segment scanning method, an ultrasound wave having a PRF of 5 kilohertz is transmitted multiple times successively along the scanning line PWD1, whereas an ultrasound wave having a PRF of 4 kilohertz is transmitted multiple times successively along the scanning line PWD2. In this situation, the transmission to the scanning line PWD1 and the transmission to the scanning line PWD2 are performed alternately multiple times each.

Further, during the segment scanning method, for example, the reflected wave from the range gate RG1 is received multiple times successively, whereas the reflected wave from the range gate RG2 is received multiple times successively. In this situation, the reception of the reflected wave from the range gate RG1 and the reception of the reflected wave from the range gate RG2 are performed alternately multiple times each. Further, for example, the data for a Doppler spectrum image at the range gate RG1 and the data for a Doppler spectrum image at the range gate RG2 are sampled alternately, in units of segments each made up of the reflected-wave data corresponding to the multiple times. During the segment scanning method, the PRFs are set to such values that make it possible to acquire the data from each of the range gates in the shortest period of time, according to the positions of the range gates RG1 and RG2.

In this situation, during the segment scanning method, while the ultrasound wave is being transmitted and received to and from the range gate RG1 successively, no ultrasound wave is transmitted and received to and from the range gate RG2. As a result, periodical data absences occur in the Doppler spectrum images at the range gates. To cope with this situation, according to the present embodiment, interpolation data is inserted into periodical data absence sections of the Doppler spectrum images at the range gates. Consequently, it is possible to inhibit degradation of the images caused by the data absences.

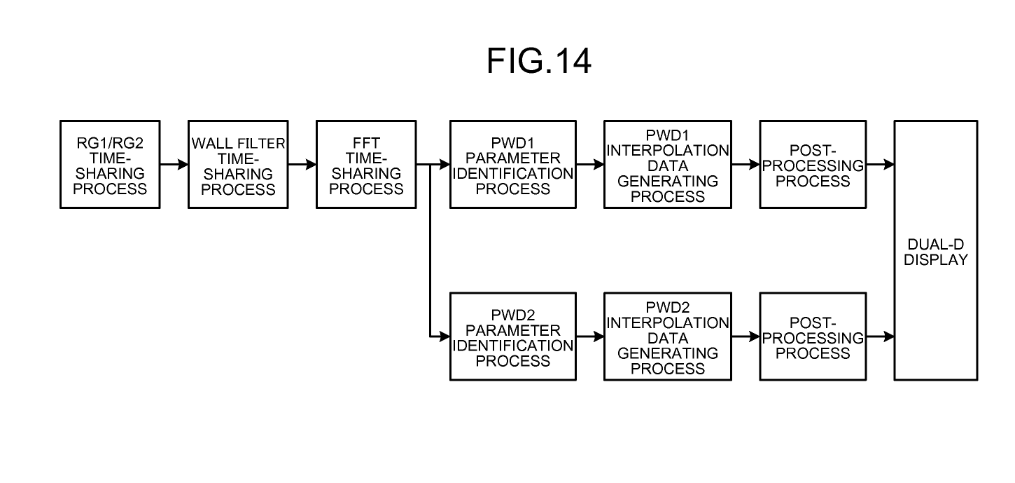

FIG. 14 is a chart of a processing flow in a segment scanning method according to the present embodiment. As shown in FIG. 14, during the segment scanning method, the Doppler processing unit 14 sequentially applies a wall filter and a Fast Fourier transformation (FFT) to the reflected-wave data from each of the range gates RG1 and RG2. In this situation, the wall filter and the fast Fourier transformation are applied as a time-sharing process. Alternatively, another arrangement is acceptable where the Doppler processing unit 14 applies a wall filter and a fast Fourier transformation to the reflected-wave data from each of the range gates RG1 and RG2 individually, like in the processing flow shown in FIG. 10, instead of applying the wall filter and the fast Fourier transformation as the time-sharing process.

Further, the Doppler processing unit 14 supplements the data absence sections in the Doppler spectrum image at the range gate RG1 with interpolation data, by applying a parameter identification process, an interpolation data generating process, and a post-processing process to the data from the range gate RG1 to which the fast Fourier transformation was applied. Similarly, the Doppler processing unit 14 also supplements the data absence sections in the Doppler spectrum image at the range gate RG2 with interpolation data, by applying a parameter identification process, an interpolation data generating process, and a post-processing process to the data from the range gate RG1 to which the fast Fourier transformation was applied. After that, from the pieces of Doppler data generated by the Doppler processing unit 14, the image generating unit 15 generates a Doppler spectrum image at the range gate RG1 and a Doppler spectrum image at the range gate RG2 and causes the display unit 2 to display the generated images (Dual-D display).

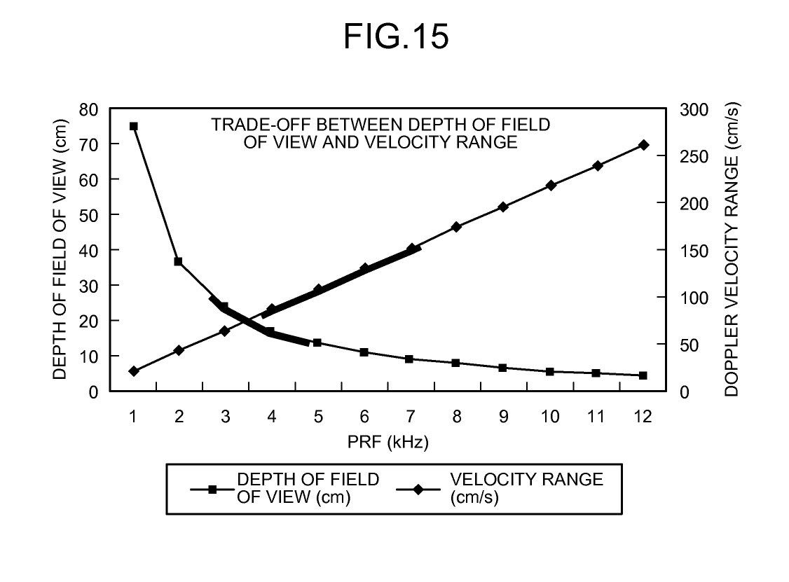

In this situation, when either the interleaved scanning method or the segment scanning method described above is performed alone, there is a possibility that it may not be possible to obtain a satisfactory Doppler spectrum image due to the sound velocity limit of the ultrasound waves, like in the conventional example. FIG. 15 is a drawing for explaining the sound velocity limit of the ultrasound waves. As shown in FIG. 15, in ultrasound waves used in ultrasound diagnosis apparatuses, the depth of the field of view, the PRF, and the Doppler velocity range are traded off against one another.

As understood also from the relationship shown in FIG. 15, when the PRF decreases, the depth of the field of view becomes deeper, whereas the Doppler velocity range becomes lower. When an interleaved scanning method is performed, the PRF decreases according to the quantity of the range gates. Thus, the velocity range of the Doppler spectrum image is lowered due to the sound velocity limit caused in this manner, and the possibility of having an aliasing phenomenon becomes higher. For this reason, when an interleaved scanning method is performed, for example, it is difficult to make a diagnosis on a fast bloodstream at a range gate set in a deep position.

To cope with this situation, according to the present embodiment, the scan switching unit 17c switches between the scanning methods so as to perform an interleaved scanning method if the distance judging unit 17b has determined that the total length of the distances is smaller than the threshold value and so as to perform a segment scanning method if the distance judging unit 17b has determined that the total length of the distances is equal to or larger than the threshold value. In other words, according to the present embodiment, when a diagnosis is to be made on the bloodstream at a range gate set in a deep position, the scanning method automatically switches from the interleaved scanning method to the segment scanning method. As a result, according to the present embodiment, even for a fast bloodstream at a range gate set in a deep position, it is possible to obtain a Doppler spectrum image having excellent image quality.

Returning to the description of FIG. 2, the measured value calculating unit 17d calculates a measured value obtained from a moving velocity represented by the first Doppler image and a moving velocity represented by the second Doppler image generated by the image generating unit 15.

For example, when the left ventricular inflow and the left ventricular outflow of the heart are the diagnosed sites, the measured value calculating unit 17d calculates various types of measured values, based on a blood flow rate represented by the Doppler spectrum image at the range gate RG1 set in the position of the left ventricular inflow and a blood flow rate represented by the Doppler spectrum image at the range gate RG2 set in the position of the left ventricular outflow. For example, as mitral-related measured values, the measured value calculating unit 17d calculates measured values such as Evel, Avel, E/A (Evel/Avel), and DcT. Further, as aortic-related measured values, the measured value calculating unit 17d calculates measured values such as VTI, VP, PPG, and MPG.

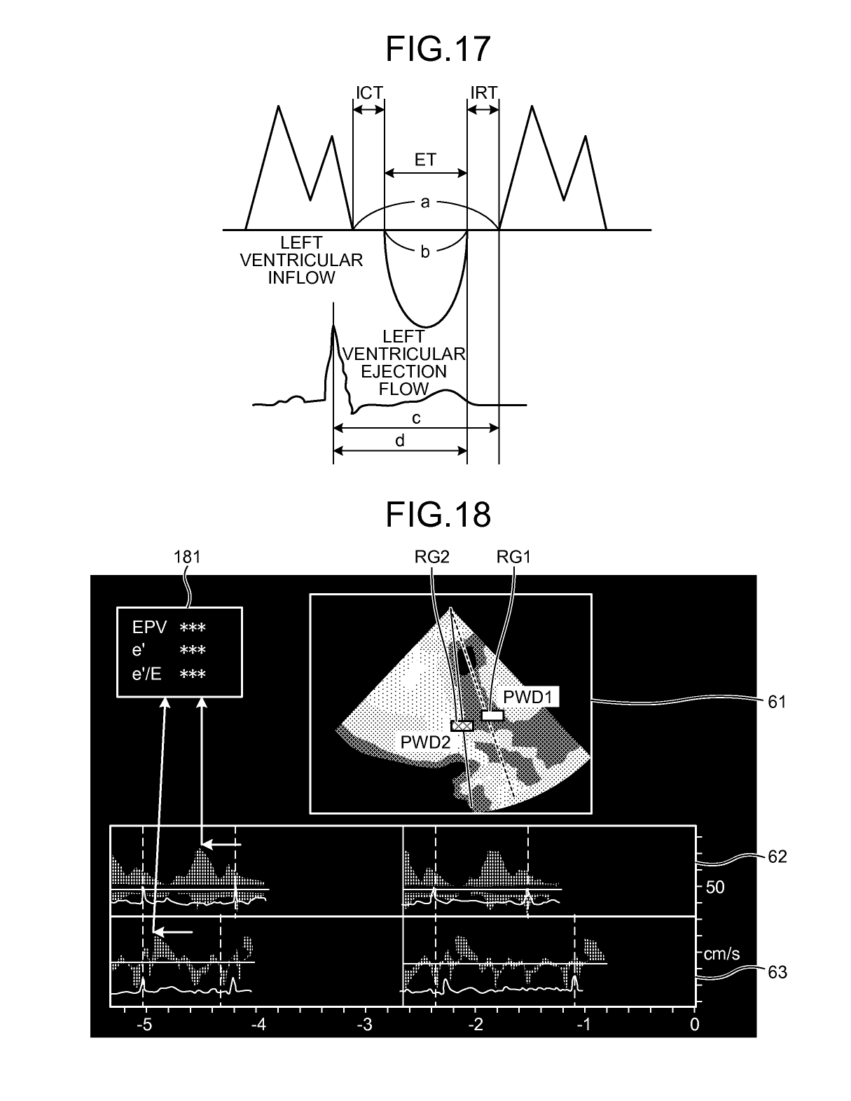

Further, as measured values related to the left ventricular inflow and the left ventricular outflow, the measured value calculating unit 17d calculates measured values such as Isovolumetric Relaxation Time (IRT) Isovolumetric Contraction Time (ICT), and T.Index. FIG. 17 is a drawing of an example of a measured value calculation performed by the measured value calculating unit 17d according to the present embodiment. For example, as shown in FIG. 17, it is possible to calculate IRT and ICT by using the formulae below, where “a” denotes a time period from the end to the start of a diastolic ventricular inflow velocity waveform, “b” denotes an Ejection Time (ET), “c” denotes a time period from an R-wave in an electrocardiogram to the start of a ventricular inflow velocity waveform, and “d” denotes a time period from an R-wave in the electrocardiogram to the end of the left ventricular ejection flow velocity waveform.

IRT=c−d

ICT=a−b−IRT

T.Index=(a−b)/b

Further, when the left ventricular inflow early diastolic filling velocity and the mitral annular motion velocity of the heart are the diagnosed sites, the measured value calculating unit 17d calculates various types of measured values based on a blood flow rate represented by the Doppler spectrum image at the range gate RG1 set in the position of the left ventricular inflow and a mitral annular motion velocity represented by the Doppler spectrum image at the range gate RG2 set in the position of the mitral valve ring. For example, the measured value calculating unit 17d calculates measured values such as EPV, e′, and e′/E. In this situation, EPV denotes a peak velocity of an E-wave in the left ventricular inflow early diastolic filling velocity waveform, whereas e′ denotes the peak value of the mitral annular motion velocity.

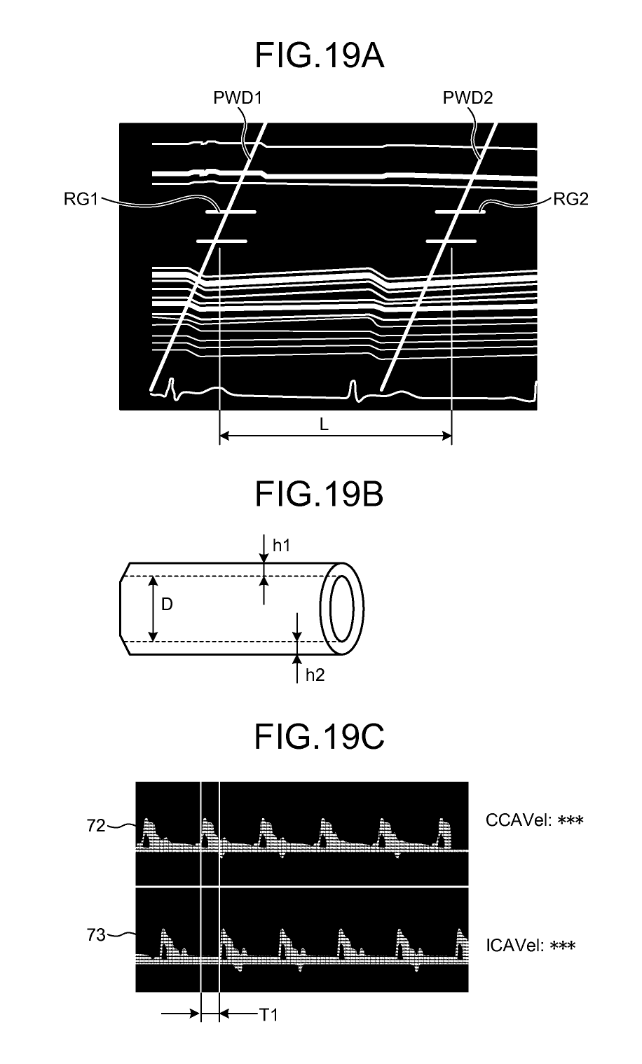

As another example, when the common carotid artery and the internal carotid artery among the carotid arteries are the diagnosed sites, the measured value calculating unit 17d calculates various types of measured values based on a B-mode image. FIGS. 19A, 19B, and 19C are drawings of examples of measured value calculations performed by the measured value calculating unit 17d according to the present embodiment. As shown in FIG. 19A, for example, the measured value calculating unit 17d calculates a distance L between a range gate RG1 and a range gate RG2. Also, for example, as shown in FIG. 19B, the measured value calculating unit 17d calculates upper and lower wall thicknesses h1 and h2 of a carotid artery, and an inside diameter D of the carotid artery.

Further, when the common carotid artery and the internal carotid artery among the carotid arteries are the diagnosed sites, the measured value calculating unit 17d calculates various types of measured values based on the blood flow rate represented by the Doppler spectrum image at the range gate RG1 set in the position of the common carotid artery and the blood flow rate represented by the Doppler spectrum image at the range gate RG2 set in the position of the internal carotid artery. For example, as shown in FIG. 19C, the measured value calculating unit 17d calculates measured values such as CCAVel, ICAVel, and T1. In this situation, CCAVel denotes the highest velocity of the CCA, whereas Icavel denotes the highest velocity of the ICA. T1 indicates the time difference between a CCA peak and an ICA peak. The measured value calculating unit 17d may also calculate a degree of arteriosclerosis E based on a pulse wave velocity C. For example, it is possible to calculate the degree of arteriosclerosis E by using Formula (I) shown below, where ρ denotes a preset value that is determined in advance for each site.

$$E = \frac{ρ \cdot D}{h} \cdot {C}^{2} \qquad ( 1 )$$

Returning to the description of FIG. 2, the measured value display unit 17e causes the display unit 2 to display the measured values calculated by the measured value calculating unit 17d.

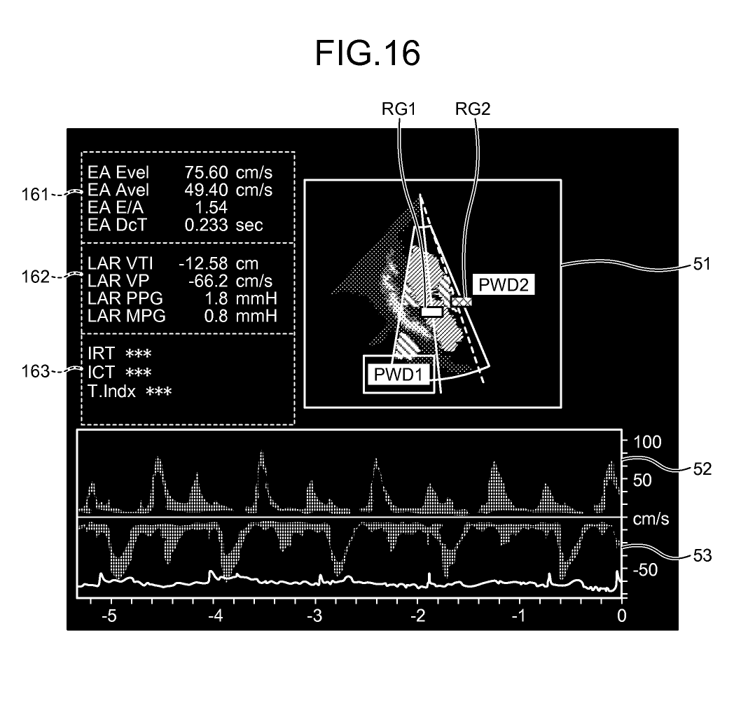

FIG. 16 is a drawing of an exemplary display of the measured values realized by the measured value display unit 17e according to the present embodiment. For example, as shown in FIG. 16, when the left ventricular inflow and the left ventricular outflow of the heart are the diagnosed sites, the measured value display unit 17e displays the measured values such as Evel, Avel, E/A (Evel/Avel), and DcT calculated by the measured value calculating unit 17d, in a display area 161 used for displaying the mitral-related measured values. Further, the measured value calculating unit 17d displays the measured values such as VTI, VP, PPG, and MPG calculated by the measured value display unit 17e, in a display area 162 used for displaying the aortic-related measured values. Further, the measured value display unit 17e displays the measured values such as IRT, ICT, and T.Index calculated by the measured value calculating unit 17d, in a display area 163 used for displaying the measured values related to the left ventricular inflow and the left ventricular outflow.

FIG. 18 is a drawing of an exemplary display of the measured values realized by the measured value display unit 17e according to the present embodiment. For example, as shown in FIG. 18, when the left ventricular inflow early diastolic filling velocity and the mitral annular motion velocity of the heart are the diagnosed sites, the measured value display unit 17e outputs the measured values such as EPV, e′, and e′/E calculated by the measured value calculating unit 17d, to a display area 181 used for displaying the measured values related to the left ventricular inflow early diastolic filling velocity and the mitral annular motion velocity.

As another example, when the common carotid artery and the internal carotid artery among the carotid arteries are the diagnosed sites, the measured value display unit 17e displays, as shown in FIG. 19C, the measured values such as CCAVel and ICAVel on the display unit 2.

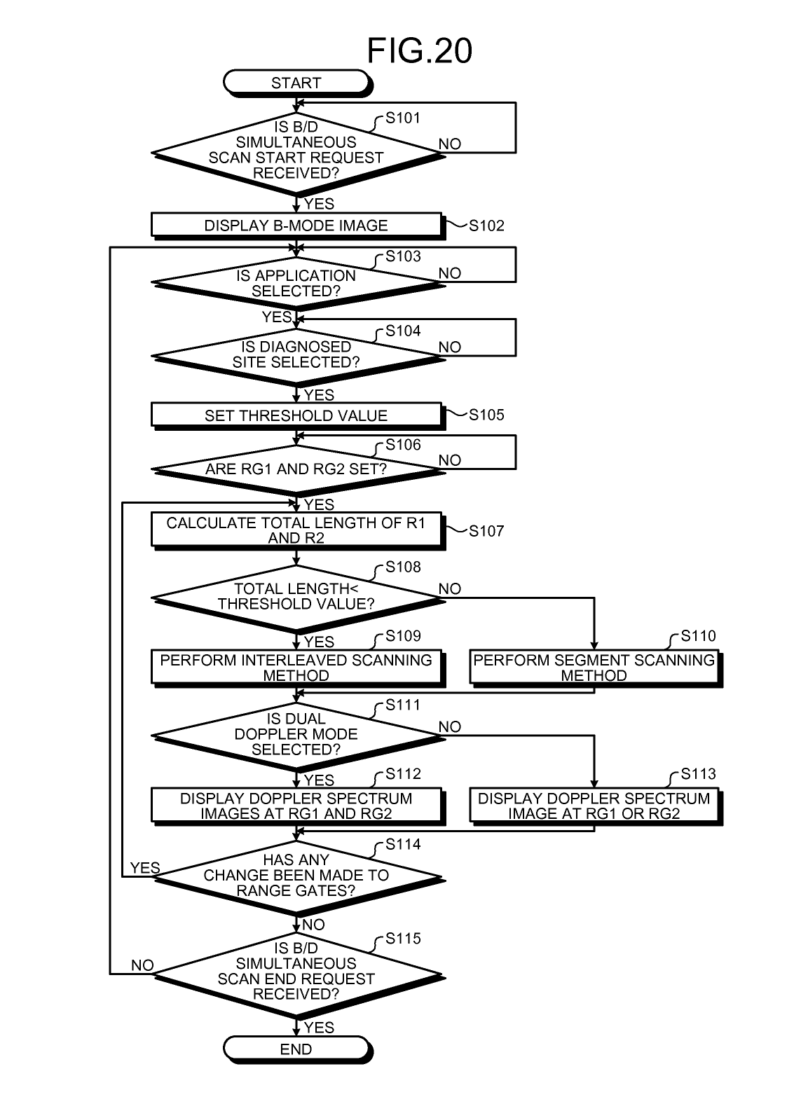

Next, a processing procedure in a B/D simultaneous scanning method performed by the ultrasound diagnosis apparatus 100 according to the present embodiment will be explained. FIG. 20 is a flowchart of the processing procedure in the B/D simultaneous scanning method performed by the ultrasound diagnosis apparatus 100 according to the present embodiment.

As shown in FIG. 20, in the ultrasound diagnosis apparatus 100 according to the present embodiment, the controlling unit 17 judges whether a B/D simultaneous scan start request is received from an operator (step S101). When a B/D simultaneous scan start request is received (step S101: Yes), the display controlling unit 17a displays a B-mode image generated by the image generating unit 15 on the display unit 2 (step S102).

After that, the display controlling unit 17a stands by until the operator selects a diagnosis-purpose application (step S103: No). When an application is selected (step S103: Yes), the display controlling unit 17a stands by until the operator selects a diagnosed site (step S104: No).

Subsequently, when a diagnosed site is selected (step S104: Yes), the distance judging unit 17b sets a threshold value used for the judgment on the switching between the scanning methods (step S105). After that, the distance judging unit 17b stands by until a range gate RG1 and a range gate RG2 are set (step S106: No).

Further, when the range gate RG1 and the range gate RG2 are set (step S106: Yes), the distance judging unit 17b calculates a total length of a distance R1 from the ultrasound probe 1 to the range gate RG1 and a distance R2 from the ultrasound probe 1 to the range gate RG2 (step S107). After that, the distance judging unit 17b judges whether the total length of the calculated distances is smaller than the threshold value (step S108).

In this situation, if the total length of the distances is smaller than the threshold value (step S108: Yes), the scan switching unit 17c switches the scanning method to the interleaved scanning method (step S109). On the contrary, if the total length of the distances is equal to or larger than the threshold value (step S108: No), the scan switching unit 17c switches the scanning method to the segment scanning method (step S110).

Subsequently, if the dual Doppler mode is being selected by the operator (step S111: Yes), the display controlling unit 17a causes the display unit 2 to display Doppler spectrum images at the range gate RG1 and the range gate RG2 (step S112). In contrast, if the dual Doppler mode is not being selected by the operator (step S111: No), the display controlling unit 17a causes the display unit 2 to display a Doppler spectrum image at the range gate RG1 or the range gate RG2 (step S113).

After that, if the operator makes any change to the range gates (step S114: Yes), the control by the controlling unit 17 returns to step S107. In this manner, as long as any change is made to the range gates, the controlling unit 17 repeatedly performs the process related to the switching between the scanning methods described above.

In contrast, if no change is made to the range gates (step S114: No), and also, no B/D simultaneous scan end request is received from the operator (step S115: No), the control of the controlling unit 17 returns to step S103. In this manner, the controlling unit 17 repeatedly performs the processes at steps S103 through S114 until a B/D simultaneous scan end request is received from the operator. Further, when a B/D simultaneous scan end request is received from the operator (step S115: Yes), the controlling unit 17 ends the process related to the B/D simultaneous scanning method.

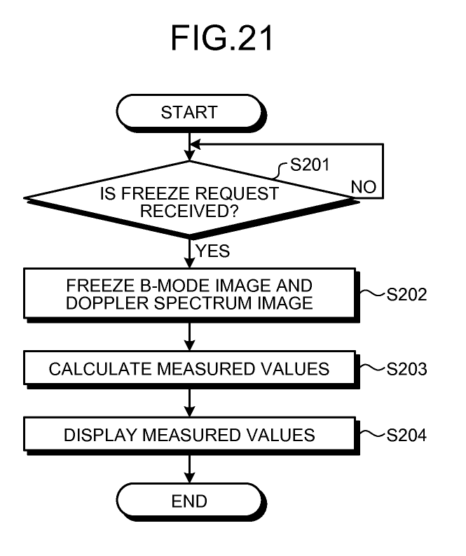

Next, a processing procedure in an automatic measuring process performed by the ultrasound diagnosis apparatus 100 according to the present embodiment will be explained. FIG. 21 is a flowchart of the processing procedure in the automatic measuring process performed by the ultrasound diagnosis apparatus 100 according to the present embodiment.

As shown in FIG. 21, in the ultrasound diagnosis apparatus 100 according to the present embodiment, the controlling unit 17 judges whether a freeze request is received from an operator (step S201). If a freeze request is received (step S201: Yes), the display controlling unit 17a freezes (stops) a B-mode image and a Doppler spectrum image (step S202).

Subsequently, the measured value calculating unit 17d calculates measured values obtained from moving velocities represented by the Doppler spectrum images generated by the image generating unit 15 (step S203). Further, the measured value display unit 17e causes the display unit 2 to display the measured values calculated by the measured value calculating unit 17d (step S204).

As explained above, the ultrasound diagnosis apparatus 100 according to the present embodiment includes the distance judging unit 17b, the scan switching unit 17c, the image generating unit 15, and the display unit 2. With respect to at least two range gates set as the bloodstream information observation sites, the distance judging unit 17b judges whether the total length of the distance from the first range gate to the ultrasound probe and the distance from the second range gate to the ultrasound probe is smaller than the threshold value. The scan switching unit 17c switches between the scanning methods so as to perform the interleaved scanning method if the total length of the distances is determined to be smaller than the threshold value and so as to perform the segment scanning method if the total length of the distances is determined to be equal to or larger than the threshold value. Based on the reflected-wave data received as a result of the segment scanning method or the interleaved scanning method, the image generating unit 15 generates the first Doppler spectrum image representing the chronological changes in the blood flow rate at the first range gate and the second Doppler spectrum image representing the chronological changes in the blood flow rate at the second range gate. The display unit 2 displays the first Doppler spectrum image and the second Doppler spectrum image generated by the image generating unit 15.

As explained above, to display the Doppler spectrum images that respectively correspond to the range gates set in the plurality of locations, the ultrasound diagnosis apparatus 100 according to the present embodiment automatically switches between the interleaved scanning method and the segment scanning method, according to the total of the depths of the range gates. With this arrangement, when a diagnosis is to be made on the bloodstream at a range gate set in a deep position, the scanning method automatically switches from the interleaved scanning method to the segment scanning method. Consequently, according to the present embodiment, it is possible to obtain Doppler spectrum images having excellent quality even for the fast bloodstream at a range gate set in a deep position. In other words, according to the present embodiment, it is possible to inhibit image quality degradation caused in the Doppler spectrum images by the sound velocity limit of the ultrasound waves.

In the embodiment described above, the scan switching unit 17c is configured to switch the scanning method to the segment scanning method when the total length of the distances from each of the range gates to the ultrasound probe 1 is determined to be equal to or larger than the threshold value. Additionally, the scan switching unit 17c may be configured, for example, to switch between the scanning methods so as to perform a segment scanning method, also if the velocity range of the first Doppler spectrum image or the second Doppler spectrum image generated by the image generating unit 15 is smaller than a predetermined velocity threshold. With this arrangement, it is possible to inhibit, with higher certainty, the image quality degradation caused in the Doppler spectrum images by the sound velocity limit of the ultrasound waves.

Further, in the embodiment described above, the distance judging unit 17b is configured to compare the total of the depths on the scanning lines in at least two of the plurality of observation sites with the predetermined threshold value; however, the exemplary embodiments are not limited to this example.

For example, another arrangement is acceptable in which the distance judging unit 17b compares a total of the depths on the scanning lines in three or more observation sites with a predetermined threshold value. In that situation, the scan switching unit 17c switches between the scanning methods so as to perform an interleaved scanning method if the total of the depth on the scanning lines in the three or more observation sites is smaller than the threshold value and so as to perform a segment scanning method if the total of the depth on the scanning lines in the three or more observation sites is equal to or larger than the threshold value.

Further, yet another arrangement is acceptable in which the distance judging unit 17b compares a depth on the scanning line in one of the plurality of observation sites with a threshold value. For example, the distance judging unit 17b may be configured to receive, from the operator, an operation to specify an observation site to be used as a reference from among the plurality of observation sites and to compare the depth on the scanning line in the observation site specified by the operator with the threshold value. In that situation, the scan switching unit 17c switches between the scanning methods so as to perform an interleaved scanning method if the depth of the observation site specified by the operator is smaller than the threshold value and so as to perform a segment scanning method if the depth in the observation site specified by the operator is equal to or larger than the threshold value.

Further, yet another arrangement is acceptable in which, for example, the distance judging unit 17b is configured to compare each of the plurality of observation sites with a threshold value, instead of using one of the observation sites as a reference. In that situation, the scan switching unit 17c performs an interleaved scanning method if the depth in at least one of the plurality of observation sites is smaller than the threshold value. Further, the scan switching unit 17c switches between the scanning methods so as to perform a segment scanning method if the depth in at least one of the plurality of observation sites is equal to or larger than the threshold value.

Further, in the embodiment described above, the scan switching unit 17c switches between the scanning methods based on the threshold value; however, the exemplary embodiments are not limited to this example.

For example, in addition to the configuration to switch between the scanning methods based on the threshold value, the scan switching unit 17c may further be configured to detect whether aliasing is occurring in the Doppler spectrum images and to switch between the scanning methods so as to perform a segment scanning method if an occurrence of aliasing is detected.

In that situation, for example, the scan switching unit 17c detects whether aliasing is occurring in the Doppler spectrum images at predetermined time intervals during the scanning method. In this situation, to detect an occurrence of aliasing, any of various types of methods may be used.

For example, the scan switching unit 17c detects a trace waveform of the maximum value of the blood flow rate, by tracing chronological changes in the maximum value of the blood flow rate, based on the Doppler data generated by the Doppler processing unit 14. The trace waveform is a waveform obtained by tracing an edge portion of a Doppler spectrum image. Further, the scan switching unit 17c calculates frequency with which each of the velocities appears based on the detected trace waveform and generates a histogram indicating a frequency distribution of the velocities. Further, the scan switching unit 17c obtains an upper limit value UL and a lower limit value LL from the histogram and determines that aliasing is occurring in the Doppler spectrum image if an absolute value |UL−LL| is larger than a first threshold value, and also, one of |UL| and |LL| is larger than a second threshold value. In this situation, the first threshold value is a value used for judging noises and the like. The second threshold value is larger than the first threshold value and is, for example, the value of a Nyquist frequency (a half of the PRF).

Subsequently, when having detected an occurrence of aliasing, the scan switching unit 17c switches to the segment scanning method at the detection point in time. As another arrangement, instead of switching between the scanning methods immediately when having detected an occurrence of aliasing in a Doppler spectrum image, the scan switching unit 17c may be configured to change the threshold value used by the distance judging unit 17b to a value smaller than the depth in one or more observation sites (the depth in at least one of the observation sites or a total of the depths in a plurality of observation sites), at the point in time when the occurrence of aliasing is detected. If the threshold value is changed in this manner, when the distance judging unit 17b compares the depth in the one or more observation sites with the threshold value, the depth in the one or more observation sites is found to be equal to or larger than the threshold value. As a result, the scan switching unit 17c switches the scanning method to the segment scanning method.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising:
a setting unit configured to set a plurality of observation sites;
a distance judging unit configured to compare a depth on a scanning line in at least one of the plurality of observation sites with a predetermined threshold value;
a scan switching unit configured to switch between scanning methods so as to perform a first scanning method by which an ultrasound wave is transmitted and received to and from each of the plurality of observation sites alternately once each if the depth on the scanning line in said at least one of the observation sites is smaller than the threshold value and so as to perform a second scanning method by which an ultrasound wave is transmitted and received to and from each of the plurality of observation sites alternately in such a manner that an ultrasound wave is transmitted and received to and from at least one of the plurality of observation sites multiple times if the depth on the scanning line in said at least one of the observation sites is equal to or larger than the threshold value;
an image generating unit configured to generate a Doppler spectrum image indicating a chronological change in a moving velocity at each of the plurality of observation sites, based on reflected-wave data received as a result of the first scanning method or the second scanning method; and
a display unit configured to display the Doppler spectrum images.

2. The ultrasound diagnosis apparatus according to claim 1, wherein
the distance judging unit compares a total of depths on scanning lines in at least two of the plurality of observation sites with a predetermined threshold, and
the scan switching unit switches between the switching methods so as to perform the first scanning method if the total of the depths on the scanning lines in said at least two of the observation sites is smaller than the threshold value and so as to perform the second scanning method if the total of the depths on the scanning lines in said at least two of the observation sites is equal to or larger than the threshold value.

3. The ultrasound diagnosis apparatus according to claim 1, further comprising:
a measured value calculating unit configured to calculate a measured value obtained from the moving velocities represented by the Doppler spectrum images; and
a measured value display unit configured to cause the display unit to display the measured value.

4. The ultrasound diagnosis apparatus according to claim 1, wherein the distance judging unit makes a judgment on the depths by setting the threshold value, based on a site on which a diagnosis is to be made or based on patient information.

5. The ultrasound diagnosis apparatus according to claim 1, wherein the scan switching unit switches between the scanning methods so as to perform the second scanning method if a velocity range of any of the Doppler spectrum images is smaller than a predetermined velocity threshold value.

6. The ultrasound diagnosis apparatus according to claim 1, wherein the scan switching unit detects whether aliasing is occurring in any of the Doppler spectrum images and, when having detected an occurrence of aliasing, the scan switching unit switches between the scanning methods so as to perform the second scanning method.

7. A method for controlling an ultrasound diagnosis apparatus, wherein
a controlling unit of the ultrasound diagnosis apparatus is configured:
to set a plurality of observation sites;
to compare a depth on a scanning line in at least one of the plurality of observation sites with a predetermined threshold value;
to switch between scanning methods so as to perform a first scanning method by which an ultrasound wave is transmitted and received to and from each of the plurality of observation sites alternately once each if the depth on the scanning line in said at least one of the observation sites is smaller than the threshold value and so as to perform a second scanning method by which an ultrasound wave is transmitted and received to and from each of the plurality of observation sites alternately in such a manner that an ultrasound wave is transmitted and received to and from at least one of the plurality of observation sites multiple times if the depth on the scanning line in said at least one of the observation sites is equal to or larger than the threshold value;
to generate a Doppler spectrum image indicating a chronological change in a moving velocity at each of the plurality of observation sites, based on reflected-wave data received as a result of the first scanning method or the second scanning method; and
to cause a display unit to display the Doppler spectrum images.
